(19) United States
(12) Patent Application Publication
(10) Pub. No.: US 2026/0159816 A1
(43) Pub. Date: Jun. 11, 2026

(54) COMPOSITION FOR INCREASING STABILITY AFTER THAWING OF FROZEN ANIMAL STEM CELL FORMULATIONS

(71) Applicants: Jeong Chan RA, Chungcheongbuk-do (KR); R Bio CO., Ltd., Seoul (KR)

(72) Inventors: Jeong Chan RA, Chungcheongbuk-do (KR); Eun-young KIM, Gyeonggi-do (KR); Hyunsoo KIM, Seoul (KR)

(21) Appl. No.: 19/416,102

(22) Filed: Dec. 11, 2025

(30) Foreign Application Priority Data
Dec. 11, 2024 (KR) 10-2024-0183278
Nov. 26, 2025 (KR) 10-2025-0182744

(51) Int. Cl. C12N 5/00

(52) U.S. Cl. CPC C12N 5/526

(57) ABSTRACT

A composition for use in cryopreservation of animal stem cells (cryopreservation formulation) and a method of cryopreserving animal stem cells using the composition are described. The animal stem cell cryopreservation solution and the cryopreservation method using the same enable long-term preservation of animal stem cells, maintain high viability after thawing, and provide the excellent effect of being directly applicable in vivo after thawing without further treatment.

### CROSS-REFERENCE TO RELATED APPLICATIONS

The priority under 35 USC § 119 of Republic of Korea Patent Application 10-2024-0183278 filed Dec. 11, 2024 and Republic of Korea Patent Application 10-2025-018-2744 filed Nov. 26, 2025 are hereby claimed, and the disclosures thereof are hereby incorporated by reference herein in their respective entireties, for all purposes.

### TECHNICAL FIELD

The present invention relates to a cryopreservation formulation for use in cryopreservation of animal stem cells and a method of cryopreserving animal stem cells using the cryopreservation formulation.

### BACKGROUND ART

The problem or limitation of currently-used animal stem cell refrigerated formulations is that the shelf life thereof is short, approximately about three days, resulting in problems in distribution. To overcome this, there is an urgent need for development of a formulation that may be stored for a long time (specifically, for at least a year or even several years) without safety issues by extending the stability period of pure and highly effective animal stem cell therapies, and that, when needed, may be directly administered in vivo after thawing without further treatment while maintaining the stability and efficacy of the product. However, an animal stem cell frozen formulation must meet safety, stability, and efficacy requirements.

Currently, the most widely used cell cryoprotectant is DMSO (dimethyl sulfoxide). DMSO is essential for freezing cells, but concentrations exceeding 5% are potentially harmful to tissues. Hence, it is necessary to reduce the amount of DMSO used, and a stem cell frozen formulation in which stability and efficacy are maintained even with reduced DMSO content needs to be developed.

Accordingly, the present inventors have endeavored to develop Cryopreserved animal Stem Cell Formulation (Ready-to-use Upon Thawing) in which the stability and efficacy of animal stem cells are maintained even at low DMSO concentrations, and ascertained that, when glucose is added to a cryopreservation composition for animal stem cell therapy (cryopreservation formulation), a cryopreservation formulation with reduced DMSO content may be prepared that maintains efficacy, ensures safety for animal use, and exhibits high product stability, thus culminating in the present invention.

### DISCLOSURE

### Technical Problem

An object of the present invention is to provide a composition for cryopreservation of animal stem cells having high animal stem cell storage stability and efficacy even at low DMSO content.

Another object of the present invention is to provide a method of cryopreserving animal stem cells using the cryopreservation formulation described above.

### Technical Solution

In order to accomplish the above objects, the present invention provides a composition for cryopreservation of animal stem cells containing glucose, DMSO, and animal serum as active ingredients.

The present invention also provides a method of cryopreserving animal stem cells, including storing and freezing animal stem cells in the cryopreservation formulation described above.

### Advantageous Effects

According to the present invention, an animal stem cell cryopreservation solution and a cryopreservation method using the same enable long-term preservation of animal stem cells, maintain high viability after thawing, and provide the excellent effect of being directly applicable in vivo after thawing without further treatment.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows results of microscopic examination of the viability of stem cells in the canine Ad-MSC fresh group and the canine Ad-MSC frozen formulation group by staining with Trypan blue;

FIG. 2 shows FACS results of the surface antigens of stem cells in the canine Ad-MSC fresh group;

FIG. 3 shows FACS results of the surface antigens of stem cells in the canine Ad-MSC frozen formulation group;

FIG. 4 shows results of Oil-Red O staining after inducing differentiation of stem cells in the canine Ad-MSC fresh group and the canine Ad-MSC frozen formulation group into adipocytes;

FIG. 5 shows results of Alizarin Red S staining after inducing differentiation of stem cells in the canine Ad-MSC fresh group and the canine Ad-MSC frozen formulation group into osteocytes;

FIG. 6 shows results of inducing chondrogenic differentiation of stem cells in the canine Ad-MSC fresh group and the canine Ad-MSC frozen formulation group; and

FIG. 7 shows results of the sterility test (direct method) of the canine Ad-MSC fresh group and the canine Ad-MSC frozen formulation group.

### MODE FOR INVENTION

Unless otherwise defined, all technical and scientific terms used herein have the same meanings as typically understood by those skilled in the art to which the present invention belongs. In general, the nomenclature used herein is well known in the art and is typical.

DMSO (dimethyl sulfoxide), currently the most widely used cell cryoprotectant, is essential for freezing cells, but if the concentration exceeds 5%, DMSO may be harmful to tissues, so there is a need to reduce content thereof. The material that the present inventors found to make this possible is glucose. The idea of using glucose was derived from hibernating animals, in which glucose serves as a crucial factor in surviving freezing conditions. Glucose travels through the blood vessels to major organs and muscles and enters the cells. This series of mechanisms prevents cellular freezing and enables the body to resume normal function after thawing, without cellular damage, as before hibernation. This means that glucose is synthesized and used as a cryoprotectant. Furthermore, glucose is a safe material in vivo.

In the present invention, a cryopreservation composition for animal stem cell therapy (cryopreservation formulation) was developed that includes glucose with reduced DMSO content, thereby maintaining efficacy and also ensuring safety in vivo and enhancing product stability. Of particular importance is not only maintaining freeze stability, but also ensuring stability and viability after thawing. The present inventors focused on this aspect in developing the formulation.

As used herein, the term “cryopreservation” means maintaining cells or tissues stably for a long period of time through freezing. Cells generally mutate at a rate of about one in ten thousand in culture, and if cell passages continue over a long period of time, the cell population changes into a different cell population from the original cell population, and in severe cases, a specific function of the cells may be lost due to passage culture. Also, infection with mycoplasma, etc. may occur during subculture. Due to these problems, cells or tissues are frozen to preserve the intrinsic characteristics thereof before they are lost, so that they may be taken out and used when needed, or tissues are cryopreserved. In particular, in order to use stem cells as a therapy, healthy stem cells must be available immediately when needed, so a method of effectively cryopreserving stem cells is particularly necessary.

Accordingly, an aspect of the present invention relates to a composition for cryopreservation of animal stem cells (cryopreservation formulation), containing glucose, DMSO, and animal serum as active ingredients.

In the cryopreservation formulation of the present invention, the glucose may be contained at a concentration of 1 to 4% (v/v), preferably at a concentration of 1.5 to 3%, more preferably at a concentration of 1.5 to 2.5%. In the cryopreservation formulation of the present invention, the DMSO may be contained at a concentration of 2 to 5% (v/v), preferably at a concentration of 3 to 4%.

In the cryopreservation formulation of the present invention, the animal serum may be contained at a concentration of 91 to 97% (v/v).

The animal serum used in the present invention may include, but is not limited to, human serum, bovine serum, fetal bovine serum, calf serum, canine serum, feline serum, rabbit serum, equine serum, goat serum, and sheep serum.

In the present invention, the animal stem cells are preferably derived from mammals and may be obtained from, for example, dogs, cattle, cats, horses, rabbits, pigs, goats, and sheep, but are not limited thereto.

An animal stem cell therapy frozen formulation according to the present invention may be administered not only by local injection (intra-articular, intramuscular, subcutaneous injection) but also intravenously.

Another aspect of the present invention relates to a method of cryopreserving animal stem cells, including storing and freezing animal stem cells in the cryopreservation composition described above.

In the present invention, the animal stem cells may be mesenchymal stem cells, embryonic stem cells, or induced pluripotent stem cells, but are not limited thereto.

The cryopreservation period of animal stem cells using the cryopreservation composition of the present invention is not limited, but is preferably 1 day to 20 years, more preferably 3 days to 10 years, even more preferably 6 months to 3 years, but is not limited thereto.

### EXAMPLES

A better understanding of the present invention may be obtained through the following examples. These examples are merely set forth to specify the present invention and are not to be construed as limiting the scope of the present invention, as will be apparent to those skilled in the art.

### Example 1: Preparation of Glucose-Containing Cryopreservation Solution and Verification of Efficacy/Stability

After culturing human-derived stem cells and harvesting the cells, 1×10⁷ cells were aliquoted, suspended in 1 ml of solution for each group, and then placed in cryovials and frozen. The composition of the cryopreservation solution by group is as follows.

TABLE 1
Group of Experiment 1

| | |
|---|---|
| Control | 5% DMSO + 95% HS |
| G1 | 5% GLU + 95% HS |
| G2 | 1% DMSO + 95% HS + 4% GLU |
| G3 | 2% DMSO + 95% HS + 3% GLU |
| G4 | 2.5% DMSO + 95% HS + 2.5% GLU |
| G5 | 3% DMSO + 95% HS + 2% GLU |
| G6 | 4% DMSO + 95% HS + 1% GLU |

[Terminology: GLU: glucose, DMSO: dimethyl sulfoxide, HS: human serum]

The purpose of developing the cryopreservation solution of the present invention is to maintain the viability by minimizing the DMSO content as much as possible, and 5% DMSO, a concentration known to be safe for the body, was used as a control.

TABLE 2
Group of Experiment 2

| | |
|---|---|
| Control | 5% DMSO + 95% HS |
| G1 | 2% GLU + 4% DMSO + 94% HS |
| G2 | 2% GLU + 3% DMSO + 95% HS |
| G3 | 2% GLU + 2.5% DMSO + 95.5% HS |
| G4 | 2% GLU + 2% DMSO + 96% HS |

In the experiment, the glucose (GLU) concentration was fixed at 2%, while the DMSO concentration was varied. After freezing, the samples were stored in a deep freezer for 3 to 10 days and then thawed, and the viability was measured over time to determine the optimal doses of DMSO and glucose. (After thawing, the viability was determined over time by storing the samples in a refrigerated state)

The results are shown below.

TABLE 3
Results of Experiment 1 (0-6 hours after
thawing, viability before freezing: 89%)

| Time after group | | Viability (%) | | | | | | |
|---|---|---|---|---|---|---|---|---|
| thawing | | 0 hr | 1 hr | 2 hr | 3 hr | 4 hr | 5 hr | 6 hr |
| Cont | 5% DMSO + | 87 | 82.2 | 88.1 | 76 | 75.7 | 72.7 | 79.3 |
| | 95% HS | | | | | | | |
| G1 | 5% GLU + | 57.6 | 19.6 | 19 | 21.3 | 18.8 | 26.5 | 15.9 |
| | 95% HS | | | | | | | |
| G2 | 1% DMSO + | 76.9 | 52.4 | 48.4 | 56 | 52.8 | 55.5 | 45.8 |
| | 4% GLU + | | | | | | | |
| | HS | | | | | | | |
| G3 | 2% DMSO + | 90.6 | 89 | 83 | 81.5 | 84.1 | 80.2 | 79.9 |
| | 3% GLU + | | | | | | | |
| | HS | | | | | | | |
| G4 | 2.5% DMSO + | 91.7 | 92.9 | 85.4 | 88.3 | 88.6 | 91.2 | 89.5 |
| | 2.5% | | | | | | | |
| | GLU + HS | | | | | | | |
| G5 | 3% DMSO + | 93.8 | 93.3 | 90.1 | 90.6 | 92.5 | 89.4 | 81.9 |
| | 2% GLU + | | | | | | | |
| | HS | | | | | | | |
| G6 | 4% DMSO + | 87.5 | 90.9 | 84.2 | 90.1 | 91.2 | 92.2 | 89.4 |
| | 1% GLU + | | | | | | | |
| | HS | | | | | | | |

TABLE 4
Results of Experiment 1 (7-120 hours after
thawing, viability before freezing: 89%)

| | | Viability (%) | | | | | | |
|---|---|---|---|---|---|---|---|---|
| Time after group | | 7 | 8 | 24 | 33 | 48 | 57 | 120 |
| thawing | | hr | hr | hr | hr | hr | hr | hr |
| Cont | 5% DMSO + | 81.2 | 82.4 | 80.3 | 79.5 | 78.1 | 68.2 | 69.9 |
| | 95% HS | | | | | | | |
| G1 | 5% GLU + | 19.7 | 20 | 19.3 | 16.8 | 16.3 | 14.6 | 10.8 |
| | 95% HS | | | | | | | |
| G2 | 1% DMSO + | 47.8 | 46 | 79.1 | 43.5 | 43.9 | 30.5 | 31.7 |
| | 4% GLU + | | | | | | | |
| | HS | | | | | | | |
| G3 | 2% DMSO + | 81.5 | 81 | 84.3 | 77.4 | 75.7 | 58.9 | 42 |
| | 3% GLU + | | | | | | | |
| | HS | | | | | | | |
| G4 | 2.5% DMSO + | 87.3 | 84.3 | 92.2 | 79.1 | 84.1 | 68.2 | 62.1 |
| | 2.5% | | | | | | | |
| | GLU + HS | | | | | | | |
| G5 | 3% DMSO + | 92.1 | 91.1 | 89.3 | 90.4 | 81.5 | 80 | 68.5 |
| | 2% GLU + | | | | | | | |
| | HS | | | | | | | |
| G6 | 4% DMSO + | 92.8 | 88.8 | 86.7 | 90.3 | 78.8 | 78.8 | 75.3 |
| | 1% GLU + | | | | | | | |
| | HS | | | | | | | |

TABLE 5
Results of Experiment 2 (0-6 hours after thawing,
viability before freezing: 92.9%)

| | | Viability (%) | | | | | | |
|---|---|---|---|---|---|---|---|---|
| Time after group | | 0 | 1 | 2 | 3 | 4 | 5 | 6 |
| thawing | | hr | hr | hr | hr | hr | hr | hr |
| Cont | 5% DMSO + | 95.9 | 94.1 | 88.7 | 94.7 | 90.2 | 89.7 | 89.2 |
| | HS | | | | | | | |
| G1 | 2% GLU + 4% | 92.7 | 90.6 | 93.5 | 86.4 | 87.6 | 87.1 | 87 |
| | DMSO + HS | | | | | | | |
| G2 | 2% GLU + 3% | 94.8 | 90.4 | 91.9 | 90.3 | 93 | 88.6 | 83.8 |
| | DMSO + HS | | | | | | | |
| G3 | 2% GLU + | 93.6 | 83.7 | 85.6 | 85.6 | 87.1 | 88.7 | 85.1 |
| | 2.5% | | | | | | | |
| | DMSO + HS | | | | | | | |
| G4 | 2% GLU + 2% | 90.5 | 82.5 | 81.5 | 80.4 | 78.3 | 73.6 | 74.6 |
| | DMSO + HS | | | | | | | |

TABLE 6
Results of Experiment 2 (7-48 hours after
thawing, viability before freezing: 92.9%)

| Time after group | | Viability (%) | | | | |
|---|---|---|---|---|---|---|
| thawing | | 7 hr | 8 hr | 24 hr | 32 hr | 48 hr |
| Cont | 5% DMSO + HS | 86.3 | 78.4 | 89.3 | 83.9 | 80.4 |
| G1 | 2% GLU + 4% DMSO + HS | 87.8 | 83.3 | 84.6 | 87.1 | 85 |
| G2 | 2% GLU + 3% DMSO + HS | 89.1 | 91.4 | 85.8 | 90.3 | 83.2 |
| G3 | 2% GLU + 2.5% DMSO + | 83.4 | 78.2 | 71.1 | 79.2 | 66.7 |
| | HS | | | | | |
| G4 | 2% GLU + 2% DMSO + HS | 70.2 | 65.6 | 70.5 | 58.2 | 50.9 |

For the cryopreservation formulation containing 2% glucose, it was confirmed that the viability of stem cells after thawing was similar to or higher than that of the control group even when the DMSO concentration was reduced.

### Example 2: Preparation of Cryopreservation Solution for Cryopreservation of Canine Adipose-Derived Stem Cells and Verification of Efficacy/Stability

Whether the glucose-containing cryopreservation solution combination (2% GLU+3% DMSO+95% serum) selected in Example 1 also had efficacy and stability in cryopreservation of animal-derived stem cells was determined.

In the present invention, the animal-derived stem cells used were canine adipose-derived stem cells, which were obtained by the following method.

Adipose tissue obtained from canine abdominal fat by liposuction was isolated and washed with saline containing antibiotics, after which the tissue was finely minced and digested at 37° C. for about 2 hours in DMEM supplemented with collagenase type 1 (1-2 mg/mL). The supernatant was suctioned, and the pellet remaining at the bottom was washed with PBS and then centrifuged at 1700 rpm for 5 minutes. After filtering through a 100 μm mesh to remove debris and washing with PBS, the cells were cultured in DMEM containing 10% FBS, growth factors, and 0.2 mM ascorbic acid. After 1 to 2 days, cells that did not attach to the culture vessel were removed, followed by subculture while the culture medium was replaced every 2 to 3 days to isolate and use canine adipose tissue-derived mesenchymal stem cells. In addition, stem cells may be obtained from canine adipose tissue by methods already known in the art.

Canine adipose-derived stem cells were cultured, and cells were harvested and then frozen at 3.5×10⁶/mL. After freezing, the cells were stored in a deep freezer for 3 to 10 days and then thawed, and viability was determined over time. After thawing in the optimal canine serum and glucose composition (3% DMSO+95% CS+2% GLU), the cells were stored in a refrigerated state and viability was determined over time (0 to 96 hours). As a control, a commonly used cell freezing solution composition (10% DMSO+20% FBS+70% medium) was used (Table 7).

Therefore, for the cryopreservation formulation containing 2% glucose, it was confirmed that the viability of stem cells after thawing was higher than or similar to that of 10% DMSO even when the DMSO concentration was reduced.

TABLE 7
Verification of storage stability of animal stem cells in
glucose-containing stem cell cryopreservation solution

| | | Viability (%) | | | | | | | | |
|---|---|---|---|---|---|---|---|---|---|---|
| | | 0 | 4 | 8 | 24 | 33 | 49 | 56 | 72 | 96 |
| | | hr | hr | hr | hr | hr | hr | hr | hr | hr |
| Control | 10% DMSO + 20% | 97.7 | 96.8 | 96 | 97.7 | 95.2 | 96.4 | 92.1 | 74.8 | 57.5 |
| | FBS + 70% | | | | | | | | | |
| | medium | | | | | | | | | |
| Experimental | 3% DMSO + 95% | 96.4 | 96.2 | 97.2 | 95 | 93.2 | 89.5 | 87.2 | 81 | 82.9 |
| group | CS + 2% GLU | | | | | | | | | |

[Terminology: GLU: glucose, DMSO: dimethyl sulfoxide, CS: canine serum, FBS: fetal bovine serum]

### Example 3: Characterization of Animal Stem Cells after Cryopreservation in Frozen Formulation

After culturing and harvesting canine adipose-derived stem cells (canine Ad-MSCs), the cells were suspended in the cryopreservation formulation (95% canine serum+2% glucose+3% DMSO) according to the present invention, placed in a cryogenic container, frozen in a deep freezer, thawed in a 37° C. water bath after 3 days, and then tested for stem cell viability, stem cell characteristics, sterility (direct method), mycoplasma detection (ALERT), and endotoxin levels (turbidimetric method). Through the above experiments, the cytotoxicity, stem cell characteristics, efficacy, and safety of the canine Ad-MSC frozen formulation were determined, confirming that stem cells frozen using the cryopreservation formulation of the present invention retained safety and efficacy, allowing direct application to cell therapy without further treatment after thawing.

TABLE 8
Experimental groups and conditions

| Group | Conditions |
|---|---|
| Canine Ad- | Canine Ad-MSCs were cultured, harvested, and used |
| MSC fresh | |
| Canine Ad- | Canine Ad-MSCs were cultured and harvested, suspended |
| MSC frozen | in a cryopreservation formulation (95% canine serum + |
| formulation | 2% glucose + 3% DMSO), placed in a cryogenic |
| | container, and frozen in a deep freezer for a certain |
| | period. After 3-10 days, the cells were thawed and |
| | used |

### 3-1: Determination of Stem Cell Viability

The viability of the canine Ad-MSC fresh group and the canine Ad-MSC frozen formulation group were assessed using Trypan blue.

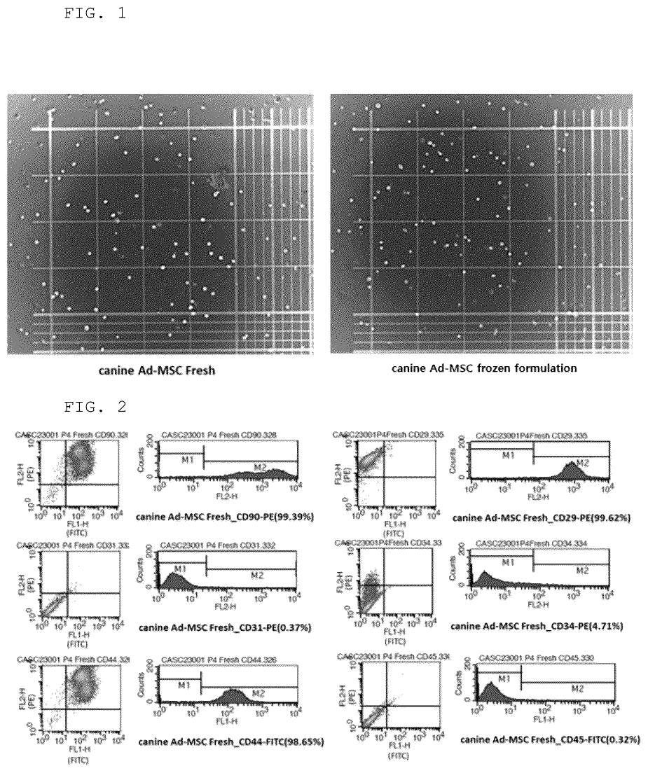

Therefore, as shown in FIG. 1 and Table 9, both groups exhibited viability of 90% or more. In conclusion, it was confirmed that the frozen formulation exhibited no cytotoxicity after thawing.

TABLE 9
Viability of canine Ad-MSC fresh & canine Ad-MSC frozen formulation
Viability (%)

| | Canine Ad- | Canine Ad-MSC |
|---|---|---|
| No. | MSC fresh | frozen formulation |
| 1 | 94.4 | 97.1 |
| 2 | 94.9 | 96.4 |
| 3 | 94.3 | 95.8 |
| SD | 0.32 | 0.65 |
| Average (%) | 94.5 | 96.4 |

### 3-2: Identification of Canine Ad-MSC Surface Antigens

The canine Ad-MSC fresh group and the canine Ad-MSC frozen formulation group were allowed to react with canine stem cell surface antigen CD markers, and surface antigens were then identified using FACS.

Stem cells were stained using antibodies conjugated with two different fluorescent materials (PE or FITC), simultaneously staining the cell surface markers CD44 and CD90, and CD29 and CD45. The remaining surface antigens were identified by single staining.

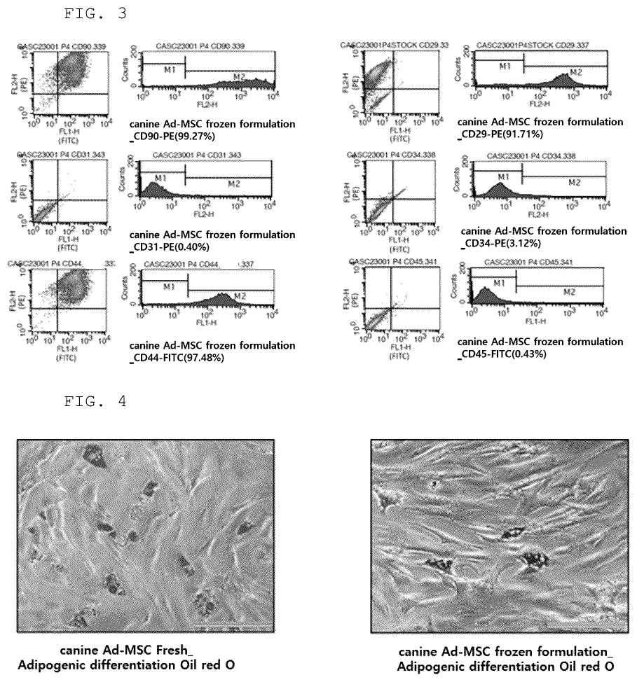

Therefore, as shown in FIGS. 2 and 3 and Table 10, the characteristics of surface antigens of canine adipose-derived mesenchymal stem cells (positive markers: CD44, CD90, CD29/negative markers: CD31, CD34, CD45) were observed in both groups.

TABLE 10
FACS results of canine Ad-MSC fresh
& canine Ad-MSC frozen formulation

| | Canine Ad-MSC | Canine Ad-MSC frozen |
|---|---|---|
| CD marker | fresh (%) | formulation (%) |
| Canine CD44 | 98.65 | 97.48 |
| Canine CD90 | 99.39 | 99.27 |
| Canine CD29 | 99.62 | 91.71 |
| Canine CD31 | 0.37 | 0.40 |
| Canine CD34 | 4.71 | 3.12 |
| Canine CD45 | 0.32 | 0.43 |

* positive %

### 3-3: Confirmation of the Differentiation Potential of Canine Ad-MSCs (Adipogenic, Osteogenic, and Chondrogenic Differentiation)

To assess the differentiation potential of canine Ad-MSCs into adipocytes, stem cells in the canine Ad-MSC fresh group and the canine Ad-MSC frozen formulation group were seeded in 12-well culture dishes, and adipogenic differentiation was induced using the OriCell™ Adipogenic Differentiation Medium For Adipose-derived Mesenchymal Stem Cells (Multipurpose) (Cyagen, GUXMD-90031), followed by Oil-Red O staining to confirm adipogenic differentiation.

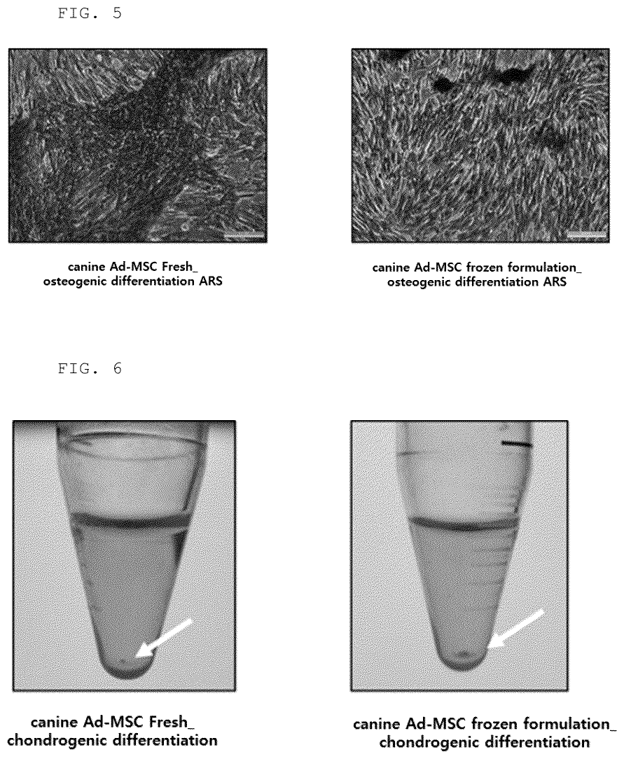

Therefore, as shown in FIG. 4, it was confirmed that adipogenic differentiation was induced in both groups.

To assess the differentiation potential of canine Ad-MSCs into osteocytes, stem cells in the canine Ad-MSC fresh group and the canine Ad-MSC frozen formulation group were seeded in 12-well culture dishes, and osteogenic differentiation was induced using the OriCell™ Osteogenic Differentiation Medium For Adipose-derived Mesenchymal Stem Cells (Multipurpose) (Cyagen, GUXMD-90021), followed by Alizarin Red staining to confirm osteogenic differentiation.

Therefore, as shown in FIG. 5, it was confirmed that osteogenic differentiation was induced in both groups.

To assess the differentiation potential of canine Ad-MSCs into chondrocytes, stem cells in the canine Ad-MSC fresh group and the canine Ad-MSC frozen formulation group were seeded in Falcon tubes, and chondrogenic differentiation was induced using the OriCell™ Chondrogenic Differentiation Medium For Adipose-derived Mesenchymal Stem Cells (Multipurpose) (Cyagen, GUXMD-90041).

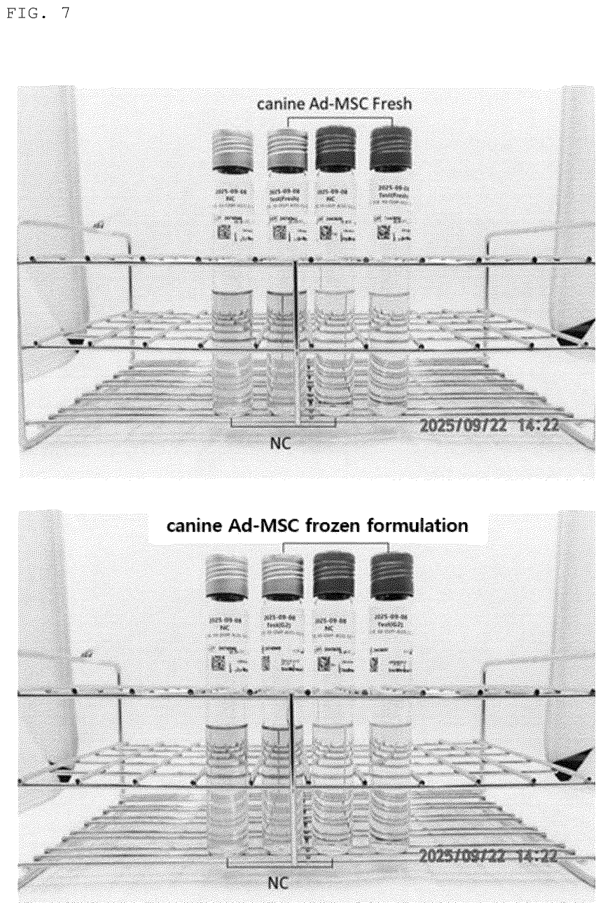

Therefore, as shown in FIG. 6, it was confirmed that chondrogenic differentiation was successfully induced in a spherical form in both groups.

### Example 4: Evaluation of Safety of Stem Cells after Cryopreservation in Frozen Formulation

The aim was to confirm the safety of the stem cells after cryopreservation in the canine Ad-MSC frozen formulation as a cell therapy that can be directly applied in vivo. To this end, tests to confirm sterility were performed using a sterility test (direct method) and a mycoplasma detection assay (ALERT method), and the absence of toxicity upon human administration was confirmed by an endotoxin assay (turbimetric method).

### 4-1: Sterility Test (Direct Method)

The sterility test involves inoculating the sample into sterile medium followed by culture in a BOD incubator, and the sample was confirmed to be sterile (negative) when no microbial growth was visually observed in the medium at the end of the culture period (14 days).

Based on results of the test using stem cells in the canine Ad-MSC fresh group and the canine Ad-MSC frozen formulation group as samples, as shown in FIG. 7 and Table 11, both samples tested negative, confirming the safety of the samples.

TABLE 11
Sterility test results of canine Ad-MSC
fresh & canine Ad-MSC frozen formulation

| Sample | Results |
|---|---|
| Canine Ad-MSC fresh | Negative |
| Canine Ad-MSC frozen formulation | Negative |

4-2: Mycoplasma Detection Assay (ALERT Method) Mycoplasma is the Smallest Bacterium Capable of Proliferation in Artificial Media, Measuring Approximately 80 to 1000 nm in size, lacking a cell wall and thus having an irregular shape, and is pleomorphic, consisting of only three layers of membranes. Contamination with mycoplasma may cause intracellular nutrient deficiency and pH changes due to accumulation of metabolites, leading to reduced yield and quality of products produced, and may also cause diseases when clinically applied.

The mycoplasma detection assay (ALERT method) involves adding mycoplasma enzyme and substrate to the sample, measuring the amount of ATP generated, and confirming that the Net B/Net A ratio of the sample is less than 0.9.

Based on results of the test using stem cells in the canine Ad-MSC fresh group and the canine Ad-MSC frozen formulation group as samples, as shown in Table 12, both samples exhibited values less than 0.9, confirming the safety of the samples.

TABLE 12
Mycoplasma detection assay results of canine Ad-
MSC fresh & canine Ad-MSC frozen formulation

| Sample | Results |
|---|---|
| Canine Ad-MSC fresh | 0.66 |
| Canine Ad-MSC frozen formulation | 0.40 |

### 4-3: Endotoxin Assay (Turbidimetric Method)

Endotoxin refers to lipopolysaccharide (LPS), which accounts for 70% of the cell wall components of Gram-negative bacteria, and is a pyrogenic substance that induces a fever response when it enters the human blood. Therefore, pharmaceuticals administered in vivo must not exceed the allowable endotoxin limit in order to be used safely.

The endotoxin assay (turbidimetric method) is based on the dose-response relationship between the endotoxin concentration and the turbidity of the reaction solution after a certain reaction period, and confirms that the endotoxin level in the sample is less than 4 EU/mL.

Based on results of the test using stem cells in the canine Ad-MSC fresh group and the canine Ad-MSC frozen formulation group as samples, as shown in Table 13, both samples exhibited values less than 4 EU/mL, confirming the safety of the samples.

TABLE 13
Endotoxin assay results of canine Ad-MSC
fresh & canine Ad-MSC frozen formulation

| Sample | Results |
|---|---|
| Canine Ad-MSC fresh | <0.200 EU/mL |
| Canine Ad-MSC frozen formulation | <0.200 EU/mL |

Having described certain parts of the present invention in detail above, it will be obvious to those skilled in the art that these specific descriptions are only preferred embodiments, and the scope of the present invention is not limited thereby. Accordingly, the substantial scope of the present invention will be defined by the appended claims and equivalents thereto.

What is claimed is:

1. A composition for cryopreservation of animal stem cells containing glucose, DMSO, and animal serum as active ingredients.

2. The composition according to claim 1, wherein the glucose is contained at a concentration of 1 to 4% (v/v).

3. The composition according to claim 1, wherein the DMSO is contained at a concentration of 2 to 5% (v/v).

4. The composition according to claim 1, wherein the animal serum is contained at a concentration of 91 to 97% (v/v).

5. The composition according to claim 1, wherein the animal serum is selected from the group consisting of human serum, bovine serum, fetal bovine serum, calf serum, canine serum, feline serum, rabbit serum, equine serum, goat serum, and sheep serum.

6. The composition according to claim 1, wherein the animal stem cells are derived from an animal selected from the group consisting of a dog, cattle, cat, horse, rabbit, pig, goat, and sheep.

7. A method of cryopreserving animal stem cells, comprising storing and freezing animal stem cells in the composition according to claim 1.

8. The method according to claim 7, wherein the animal stem cells are mesenchymal stem cells, embryonic stem cells, or induced pluripotent stem cells.

9. The method according to claim 7, wherein the animal stem cells are derived from an animal selected from the group consisting of a dog, cattle, cat, horse, rabbit, pig, goat, and sheep.
